(19) United States
(12) Patent Application Publication
(10) Pub. No.: US 2018/0214332 A1
(43) Pub. Date: Aug. 2, 2018

(54) MAXILLOFACIAL REHABILITATION DEVICE

(71) Applicants: Michael S. YOUNG, College Station, TX (US); Ritesh BHATTACHARJEE, Irving, TX (US); Raktim BHATTACHARYA, College Station, TX (US); Brandon FORESMAN, Irving, TX (US); THE TEXAS A&M UNIVERSITY SYSTEM, College Station, TX (US)

(72) Inventors: Michael S. Young, College Station, TX (US); Ritesh Shattacharjee, Irving, TX (US); Raktim Bhattacharya, College Station, TX (US); Brandon Foresman, Irving, TX (US)

(73) Assignee: THE TEXAS A&M UNIVERSITY SYSTEM, College Station, TX (US)

(21) Appl. No.: 15/746,310

(22) PCT Filed: Jul. 20, 2016
(86) PCT No.: PCT/US2016/043095

§ 371 Date: Jan. 19, 2018

Related U.S. Application Data
(60) Provisional application No. 62/194,602, filed on Jul. 20, 2015.

(51) Int. Cl. A61H 1/00

(52) U.S. Cl. CPC A61H 1/00; G16H 40/63; A61H 2201/165; A61H 2201/5043; A61H 2201/5097; A61H 2205/026; A61H 2201/1664; A61H 2201/149; A61H 2201/5007; A61H 2201/5038; A61H 2201/5061; A61H 2201/5064; A61H 2201/5079; A61H 2201/1215; A61H 2201/1238; A61H 2201/1607

(57) ABSTRACT

The disclosure includes a maxillofacial rehabilitation device, comprising a frame and a dynamic actuation mechanism coupled to the frame. The frame can be configured to attach the device to the head of a user. The dynamic actuation mechanism can be configured to contact the mandible of the user, receive control signals from a controller, and move the mandible of the user in the sagittal plane and the vertical plane based on the control signals.

### RELATED APPLICATIONS

This application is the National Stage of International Application No. PCT/US2016/43095, filed Jul. 20, 2016; which claims priority to U.S. Provisional Patent Application 62/194,602 filed on Jul. 20, 2015 which are both specifically incorporated by reference in their entirety herein.

### FIELD

The disclosure relates generally to medical devices. The disclosure relates specifically to maxillofacial rehabilitation devices.

### BACKGROUND

Traumatic events to and developmental defects in the jaw can cause movement abnormalities that inhibit the regular function of the mouth. For example, cancer patients undergoing chemotherapy in the jaw can develop fibrosis in the muscles of the jaw, leading to a condition called trismus. Trismus is characterized by limited movement of the mouth, and can worsen over time, depending on the cause of the condition. Other diseases or events that can inhibit mouth function include temporomandibular joint (TMJ) disorders, tumors, arthritis, and fractures. Due to the variable nature of these conditions, the injury profile of the patient can be asymmetrical.

Researchers have attempted to replicate mastication with a robotic device and create a device to rehabilitate TMJ disorders. Almost all of these devices are not wearable. One device that is wearable is not ergonomic, does not autonomously adjust to the patient's jaw, only has two degrees of freedom, and does not provide feedback to the user. The device has a single motor and must be adjusted by hand.

At this point, there are five commercially available devices used to rehabilitate jaw abnormalities particularly relating to jaw stiffness or trismus. These devices are the Orastretch, Therabite, EZ Flex Exerciser, Dynasplint Oral, and the Therapacer. The Orastretch, Therabite, and Dynasplint are devices that attempt to provide passive sustained opening to the mouth using a hand operated mechanical apparatus. They engage the upper and lower jaw using various mouth trays that engage the upper and lower arches of the jaw and teeth. Rehabilitation is provided using hand powered expansion of the arms of the apparatus which expand the jaw. There are multiple issues with this approach. First, these devices require at least 12 mm of mouth opening to be usable. This degree of mouth opening may not be possible in many people suffering from this condition. Second, because the exercise regimen works only on continuous use, activation of the device using hand movements causes fatigue among users. Third, constant opening causes pooling of saliva in the mouth, leading to drooling. Fourth, the device contraption engages the front arch teeth from 1st premolar to 1st premolar on each side, both upper and lower. This causes increased forces on the TMJ instead of the muscles of mastication. These forces increase the difficulty in mastication, reduce effectiveness of therapy, and exacerbate joint problems. The Dynasplint system uses a clamp screw apparatus to facilitate jaw opening. The Dynasplint uses a customized jaw registration using impressions of the upper and lower jaw specific to each jaw. The clamp is activated using a screw system. Since the device is very heavy, it can be operated hands free only if counterweights are used. The EZ Flex Exerciser uses a plastic mouth engagement system engaging the upper and lower front teeth. It uses expansion and contraction of a balloon apparatus to facilitate jaw exercise. Expansion of the balloon is done using a syringe valve system that is regulated by the user. This system has the same structural issue as the Therabite and Orastretch systems. Like the Therabite and Orastretch, the EZ Flex only engages the front teeth. Engaging only the front teeth leads to increased loading of the Temporomandibular Joint, instead of uniform force distribution across the muscles and joint. The Orastretch is the only automated device. It uses a computer controlled system to provide passive movement of the jaw. It engages the jaw similar to the Therabite, and provides unidirectional downward motion for the lower jaw while engaging the upper jaw. The Therapacer is extremely painful due to the way it engages the teeth and mitigates progression of the trismus as opposed to treating it.

In “A Novel Wearable Assistive Device for Jaw Motion Disability Rehabilitation” by Wang describes a 2 degree of freedom (“DOF”) wearable system to treat trismus. The system utilizes 2 motors and 2 cross bars to engage the TMJ in only the sagittal plane. This is an improvement from the devices above but requires precise adjustment by the user to match the specific needs of the patient. It does not utilize impedance control or system identification methods to optimize the therapy. It also does not implement bilateral loading since the bottom bite plate is a single bar across the teeth. The device is non-ergonomic and requires excessive parts to mimic the trajectory of the mandible.

It would be advantageous to have a rehabilitation device that utilizes impedance control, methods to optimize therapy, and has six degrees of freedom.

### SUMMARY

The disclosure includes a maxillofacial rehabilitation device, comprising a frame and a dynamic actuation mechanism coupled to the frame. The frame can be configured to attach the device to the head of a user. The dynamic actuation mechanism can be configured to contact the mandible of the user, receive control signals from a controller, and move the mandible of the user in the sagittal plane and the vertical plane based on the control signals.

An embodiment of the disclosure is a maxillofacial rehabilitation device, comprising: a frame, wherein the frame is used to attach the device to the head of a user; a dynamic actuation mechanism coupled to the frame wherein the dynamic actuation mechanism contacts the mandible of the user; receives control signals from a controller; and moves the mandible of the user in the sagittal plane and the vertical plane based on the control signals. In an embodiment, the frame comprises a headset having a left end coupled to a left base plate and a right end coupled to a right base plate. In an embodiment, the left base plate fits over a left ear of the user and the right base plate fits over a right ear of the user. In an embodiment, the device further comprises a maxilla plate coupled to the left base plate and the right base plate wherein the maxilla plate contacts the maxilla of the user. In an embodiment, the maxilla plate contacts the inside of the mouth of the user. the dynamic actuation mechanism comprises a left mandible plate coupled to a left mandible support; a right mandible plate coupled to a right mandible support; a left actuator mechanism coupled to the left base plate and the left mandible support and wherein the left actuator mechanism moves the left mandible plate; and a right actuator mechanism coupled to the right base plate and the right mandible support and wherein the right actuator mechanism moves the right mandible plate. In an embodiment, the left actuator mechanism further comprises a first left actuator, wherein the first left actuator elevates and depresses the left mandible plate; a second left actuator, wherein the second left actuator protrudes and retrudes the left mandible plate; and the right actuator mechanism further comprises a first right actuator, wherein the first right actuator elevates and depresses the right mandible plate; a second right actuator, wherein the second right actuator protrudes and retrudes the right mandible plate. In an embodiment, the first left actuator is fixed to the left base plate; the second left actuator is coupled to the left base plate with a left vertical pivot joint; the first right actuator is fixed to the right base plate; and the second right actuator is coupled to the right base plate with a right vertical pivot joint. In an embodiment, the dynamic actuation mechanism moves the left mandible plate and the right mandible plate in the transverse plane. In an embodiment, the frame further comprises a left transverse plate coupled to the left base plate; a right transverse plate coupled to the right base plate; the left actuator mechanism further comprises a third left actuator to move the left mandible plate in the transverse plane; the right actuator mechanism further comprises a third right actuator to move the right mandible plate in the transverse plane; the first left actuator is coupled to the left transverse plate with a left transverse pivot joint; the second left actuator is coupled to the left transverse plate with a left vertical pivot joint and a left transverse pivot structure; the first right actuator is coupled to the right base plate with a right transverse pivot joint; and the second right actuator is coupled to the right transverse plate with a right vertical pivot joint and a right transverse pivot structure. In an embodiment, the first left actuator and the first right actuator move the mandible plate in the transverse plane. In an embodiment, the left actuator mechanism further comprises: a left pulley set coupled to the left base plate and the left mandible support; a left remote actuator coupled to the left pulley set with left cables, wherein the left cables are attached to the left mandible support; and a left support system coupled to the left base plate and to guide the left mandible support; and the right actuator mechanism further comprises: a right pulley set coupled to the right base plate and the right mandible support; a right remote actuator coupled to the right pulley set with right cables, wherein the right cables are attached to the left mandible support; and a right support system coupled to the right base plate and to guide the right mandible support. In an embodiment, the device further comprises the controller sending control signals to the dynamic actuation mechanism. In an embodiment, the controller senses user characteristics and determines a profile for the user based on the user characteristics; and determines control signals based on the profile. In an embodiment, the user characteristics include mandible force and displacement information; the control signals include left mandible plate and right mandible plate displacement, force, and velocity information. In an embodiment, the controller updates the profile based on one or more changes in the user characteristics. In an embodiment, the left actuator mechanism further comprises: a left position sensor coupled to the controller to sense the position of the left mandible support; a left force sensor coupled to the controller to sense the force on the left mandible support; and a left velocity sensor coupled to the controller to sense the velocity of the left mandible support; and the right actuator mechanism further comprises: a right position sensor coupled to the controller to sense the position of the right mandible support; a right force sensor coupled to the controller to sense the force on the right mandible support; and a right velocity sensor coupled to the controller to sense the velocity of the right mandible support. In an embodiment, the controller comprises a computing station connected to the device. In an embodiment, the controller comprises an external user display connected to the device. In an embodiment, the device further comprises a wireless transmission apparatus for sending and receiving signals from the device.

The foregoing has outlined rather broadly the features of the present disclosure in order that the detailed description that follows can be better understood. Additional features and advantages of the disclosure will be described hereinafter, which form the subject of the claims.

### BRIEF DESCRIPTION OF THE DRAWINGS

In order that the manner in which the above-recited and other enhancements and objects of the disclosure are obtained, a more particular description of the disclosure briefly described above will be rendered by reference to specific embodiments thereof which are illustrated in the appended drawings. Understanding that these drawings depict only typical embodiments of the disclosure and are therefore not to be considered limiting of its scope, the disclosure will be described with additional specificity and detail through the use of the accompanying drawings in which:

FIG. 1 is an exemplary diagram of a maxillofacial rehabilitation device having an internal controller, according to embodiments of the disclosure. A sagittal plane device with bilateral loading is depicted.

FIG. 2A and FIG. 2B are exemplary diagrams of a maxillofacial rehabilitation device having a local actuation mechanism, external mandible engagement, and three planes (sagittal, vertical, and transverse) of mandible movement, according to embodiments of the disclosure. A device with six degrees of freedom is depicted.

FIG. 3 is an exemplary diagram of a maxillofacial rehabilitation device having a remote actuation mechanism and two planes (sagittal and vertical) of mandible movement, according to embodiments of the disclosure. A sagittal plane design with Bowden cables is depicted.

FIG. 4 is an exemplary flow diagram of a process for controlling a maxillofacial rehabilitation device, according to embodiments of the disclosure.

FIG. 5 is an exemplary flow diagram of a process for use of the maxillofacial rehabilitation device.

FIG. 6 is an exemplary diagram of a maxillofacial rehabilitation device.

FIG. 7 is an exemplary diagram of a maxillofacial rehabilitation device.

FIG. 8 is an exemplary diagram of a maxillofacial rehabilitation device.

FIG. 9 is an exemplary diagram of a maxillofacial rehabilitation device.

FIG. 10 is an exemplary diagram of a maxillofacial rehabilitation device.

FIG. 11 is an exemplary diagram of a maxillofacial rehabilitation device.

FIG. 12 is an exemplary diagram of a maxillofacial rehabilitation device having a pulley-like embodiment with motors in the external casing.

FIG. 13 is an exemplary diagram of a maxillofacial rehabilitation device having a headphone design without mouthpieces.

FIG. 14 is an exemplary diagram of a maxillofacial rehabilitation device having possible six degrees of freedom.

FIG. 15 is a close-up of a ball joint of FIG. 14.

### DETAILED DESCRIPTION

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings making apparent to those skilled in the art how the several forms of the disclosure can be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary 3^{rd} Edition.

There are 250,000 people living in the US with a condition called trismus. This condition is a consequence of the treatment for head and neck cancers, trauma, surgery, etc. and is defined as limited mouth opening. Current modalities to treat this disease are painful, expensive, and are unable to provide the necessary physiotherapeutic results. Thus, we are developing a wearable, autonomous solution that increases patient compliance, improves efficacy of care, and ultimately provides a higher quality of life for cancer patients/survivors.

To improve the functioning of the jaw in patients with reduced motion, the tissues of the jaw can be exercised by moving the jaw through a greater range of motion or providing resistance against the jaw. Certain devices can stretch the jaw in one or two directions of motion. However, the jaw operates in six directions of motion or degrees of freedom (DOF). Further, those devices cannot accommodate the unique injury and treatment profile of a patient.

The path of the jaw opening is not linear or two dimensional. The path of the jaw opening functions with six degrees of freedom. Some of the current devices, like the Therabite and Orastretch, try to incorporate a curved path of opening similar to the TMJ. But in reality, the TMJ path is unique for each individual. This is because each joint and muscle system for every human is unique and constantly changes with age and growth. Ease of use is also a major concern that leads to a significant lack in patient compliance.

According to embodiments of the disclosure, a maxillofacial rehabilitation device can facilitate treatment of jaw conditions requiring personalized exercise and resistance. The maxillofacial device can be secured to a user's head to allow the user greater freedom of movement. A dynamic actuation mechanism can assist resistance or movement of the user's mandible along vertical, sagittal, and/or transverse planes. The dynamic actuation mechanism can be controlled to provide personalized identification, feedback, and treatment for the user.

The technology utilizes a series of actuators in conjunction with an engagement apparatus for the mouth to exercise the tissues in an optimal method specific to that patient. In an embodiment, the device can be wearable (like headphones) and have 2 small extensions on either side to engage the jaw. In various embodiments, the device can involve hydraulic pumps, Bowden cables, and/or springs that may sit on the shoulders or sit in the mouth. In an embodiment, algorithms are used to control the therapy and provide feedback to the patient/physician. In an embodiment, the device configuration can be modified depending on the patient population. In an embodiment, the algorithms can be applied to other therapies. In an embodiment, the device can be used on adults. In an embodiment, the device can be used on children.

In an embodiment, the device will administer slow, passive therapy to slowly stretch the tissues. In an embodiment, the device also has the capability to provide resistance to bite force to help regenerate the muscles. These features are also applicable to teaching oral motor patterns for children with Cerebral Palsy, stroke patients, or any form of facial paralysis involving the Temporomandibular Joint and/or muscles of mastication. In an embodiment, the device can be used after radiation therapy.

In an embodiment, the device can improve the standard of care for patients with any sort of TMJ Disorder, who lack oral function, or cannot open their mouth. Since the mouth and face are the gateway to basic physiologic functions such as eating and drinking, the device can improve overall nutrition. In an embodiment, the device can also provide a better field of vision for physicians, dentists, and speech therapists with the possibility to promote therapeutic tissue remodeling. For example, a 4.5-hour dental procedure can become a 30-minute dental procedure for patients with TMJ Disorder, who lack oral function, or cannot open their mouth. This is an improvement for patients, physicians, and insurance companies. In addition, trismus patients are an underserved population in general and the device can improve their quality of life. In an embodiment, the device can be used for other patient groups than those with trismus. Historically, there has been a lack of research for treating trismus and the research that was performed did not properly consider the patient's needs. Treatment for trismus can be lifelong and need to be administered for long periods of time, such at 1-2 hours at a time. It is inconvenient for the patient to go to the doctor's office daily to use a machine for 1-2 hours. Previous devices did not provide autonomous treatment, required adjustment, and were not ergonomic for the user. The device disclosed herein allows the patient to perform other tasks while using the device such as reading, working on a computer, or watching television. The device disclosed herein uses estimation and optimal control techniques to provide a cost-effective and user-friendly method of administering customized treatment. In an embodiment, the device disclosed herein utilizes impedance control to treat the temporomandibular joint disorders.

The maxillofacial rehabilitation device can include a frame configured to attach the device to a user's head. The frame can include a headset and two base plates, each base plate attached to an end of the headset. The headset and base plates can be designed to mimic the fit of ergonomic devices worn on a user's head, such as headphones. The frame can also include a maxilla support mechanism to secure the device to the maxilla of the user. The maxilla support mechanism can include left and right maxilla support structures coupled to the base plates at one end and one or more maxilla plates at the other end. The maxilla plates can fit inside or outside the mouth. Alternatively, the frame can be coupled to an additional support at a location on the head other than the maxilla.

A dynamic actuation mechanism can be coupled to the frame and configured to contact the mandible of the user, receive control signals from a controller, and move the mandible of the user in at least the sagittal plane and the vertical plane. The dynamic actuation mechanism can have a left actuator mechanism and a right actuator mechanism, each for controlling the resistance on the mandible on each side of the head. Each actuator mechanism can contact the mandible through a mandible support mechanism and can be operated independently. The mandible support mechanism can include a mandible support and a mandible plate.

The dynamic actuation mechanism can move the mandible of the user in the sagittal, vertical, and/or transverse planes. These movements can include, but are not limited to, opening and closing of the mouth, movement of the jaw from side to side, and movement of the jaw back and forth. Many movements of the mandible can occur in multiple planes. For example, a person's jaw is hinged and can follow a unique trajectory that includes a vertical component and a sagittal component.

In some embodiments, each actuator mechanism can utilize linear actuators and directional joints to achieve mandible movement. These linear actuators can extend and retract in particular planes of motion to achieve the desired positioning of the mandible supports. For example, a linear actuator operating in a sagittal plane can pivot against the frame of the device and extend and retract the mandible support to achieve protrusion, retrusion, elevation, and depression of the jaw. A linear actuator operating in a vertical plane can extend downward. Additional actuators can be added, such as a linear actuator for transverse movement.

In other embodiments, each actuator mechanism can utilize pulleys and an external actuating force to achieve mandible movement. The pulleys and joints can act as guides for a drive mechanism that includes an external actuation mechanism, cables, and the mandible support. A user can still freely move his head, with the cables providing adaptive support.

A controller can be coupled to the dynamic actuation mechanism to control movement of the mandible. The controller can be coupled to sensors on the device to sense user characteristics that include a force sensor for sensing force on the mandible support, a trajectory sensor for sensing movement of the mandible support, and a position sensor for sensing the position and displacement of the mandible support. These sensors can transmit signals to the controller. The controller can create a profile of the user based on the user's characteristics. For example, if a user has significant atrophy of the muscles of her face, her profile will show the strength of her jaw muscles, the extent to which she can open her mouth, and the trajectory of her jaw, among other characteristics. The controller can determine control signals based on the user profile, such as the force to be exerted, the path of exertion, the optimal trajectory, the speed of movement, and the limits of movement of the mandible support.

The disclosed device has the ability to autonomously adjust to the individual patient's optimal mouth opening trajectory. It does this by sensing the displacement, velocity, and force required of the movement by the device acting on the mandible and maxilla. With this information, the system will use system identification techniques for comparison with a biomechanical model to determine which muscles and to what degree the muscles are fibrosed. Thus, an optimal trajectory can be determined.

The controller can also collect and provide feedback during operation of the device. As a user profile changes, the controller can update the user profile to reflect the changes and modify the treatment parameters based on the changes. The controller can be operated as a single unit or can have its operations performed discretely, such as on a cloud computing system or remote workstation.

For the actual therapy, the device will move about a standard trajectory to collect initial data. The rate of movement will occur at a range of 0.1 mm/hr to 30 mm/hr. The device has the capability to move at a faster rate, but scientific data indicates that a slower rate has been more effective. Patient safety comes into account as well with faster rates.

To account for all variations in the human maxilla, mandible, teeth forms, TMJ, and/or mouth opening, the device has the capability to move in all 6 degrees of freedom. This also allows for complicated movement patterns such as mastication.

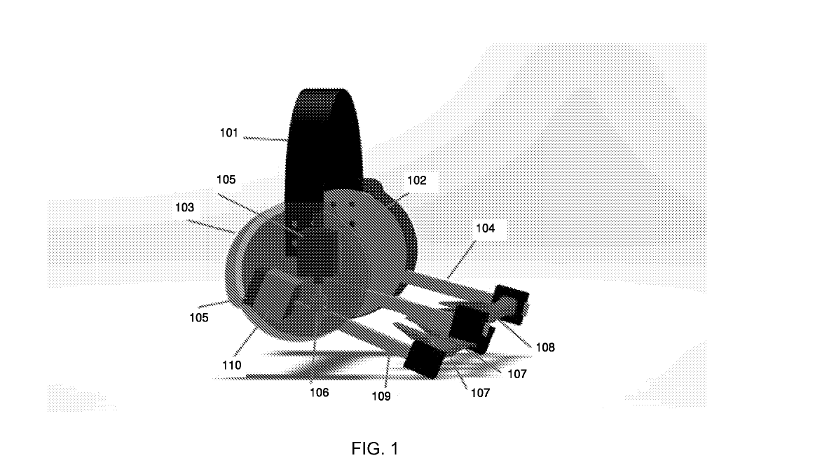

For the simpler embodiment that operates in the sagittal plane, FIG. 1, the device can still customize the loading on either side of the mandible and adjust the trajectory with the extra motor.

To facilitate patient compliance, the device is wearable and aesthetically pleasing. By using a headphone type design, this would more easily be worn in public or a work setting as needed.

The autonomous therapy and logging of data monitors constant progress for the patient and physician. This device can be used for, but not limited to, the treatment of trismus, TMJ disorders, and to teach patients how to chew.

In this device, the stepper motors can be replaced by various actuators that include but are not limited to hydraulic, pneumatic, and solenoid actuators. It can also utilize springs for resistance training in conjunction with the motors.

In an embodiment, the device is wearable and actuates the mandible in multiple degrees of freedom in order to stretch and exercise the muscles of mastication, rehabilitate the temporomandibular joint, and/or teach proper mastication techniques. In an embodiment, the device engages the mandible intraorally. In an embodiment, the device engages the mandible externally and the maxilla externally. In an embodiment, the device allows for modular attachments to incorporate other forms of muscular treatment and stimulation, and programmable exercises for individual customization. In an embodiment, the device actuates autonomously based on optimal trajectory determined by system identification and optimization techniques. In an embodiment, the device engages intraorally with upper and lower mouth plates. In an embodiment, the mouth plates are in two portions, right and left, to allow for bilateral loading that can compensate for irregularities in the patient's TMJ, including but not limited to cants, teeth form, and uneven fibrosis. In an embodiment, the device comprises two motor casings. In an embodiment, the motor casings contain 1-3 motors each. In an embodiment, the motor casings contain 1-3 position, velocity, and/or force sensors each. In an embodiment, the motor casings contain a series of pulleys, cables, and Bowden cables attached to an external motor box. In an embodiment, the mouth pieces in are connected to two bars connecting to the two motors. In an embodiment, the motors can move multiple degrees of freedom allowing the mouth plates to open the jaw vertically, imitating the natural movement of the mandibular joint, but also, horizontally, diagonally, in a circular motion, and in any other reasonable motion as may be necessary for treatment. In an embodiment, the device contains a headset connected to the motor casings.

In an embodiment, the device connects to an external user display and computing station. In an embodiment, the computing station contains a microprocessor, memory, and necessary circuitry to connect the system. In an embodiment, the computing station contains a wireless transmission apparatus to send data to the cloud or other computing platform.

FIG. 1 is an exemplary diagram of a maxillofacial rehabilitation device having a local actuation mechanism, internal mandible engagement, and two planes (sagittal and vertical) of mandible movement, according to embodiments of the disclosure. A headset 101 can be coupled to a left base plate and a right base plate 102. In an embodiment, it is attached rigidly. A top mouth plate 108 can be coupled to the base plates 102 by maxilla supports 104. The headset 101, base plates 102, maxilla supports 104, and top mouth plate 108 can be coupled rigidly to form the device frame.

The base plate rigidly attaches to the vertical linear actuator, 105, and the outer casing of the bearing that attaches to the rotating motor casing, 110. The motor casing holds the other linear actuator, 105, whose push rod attaches to the sliding bottom rod 109, with a bearing. To achieve mouth opening, the vertical linear actuator will “push” down on the joint, 106, or extend to move the other motor, sliding rod, and bottom side mouth piece that are rigidly attached. Oppositely, the vertical linear actuator will retract to close the jaw. To move the mandible forward and backward, the rotating motor will extend or retract depending on the desired movement. The two movements combined can yield any movement in the sagittal plane. The left and right bottom are separate to provide bilateral loading of the mandible in order to compensate for any discrepancies including but not limited to muscle stiffness, teeth form, sensitivity, and cant.

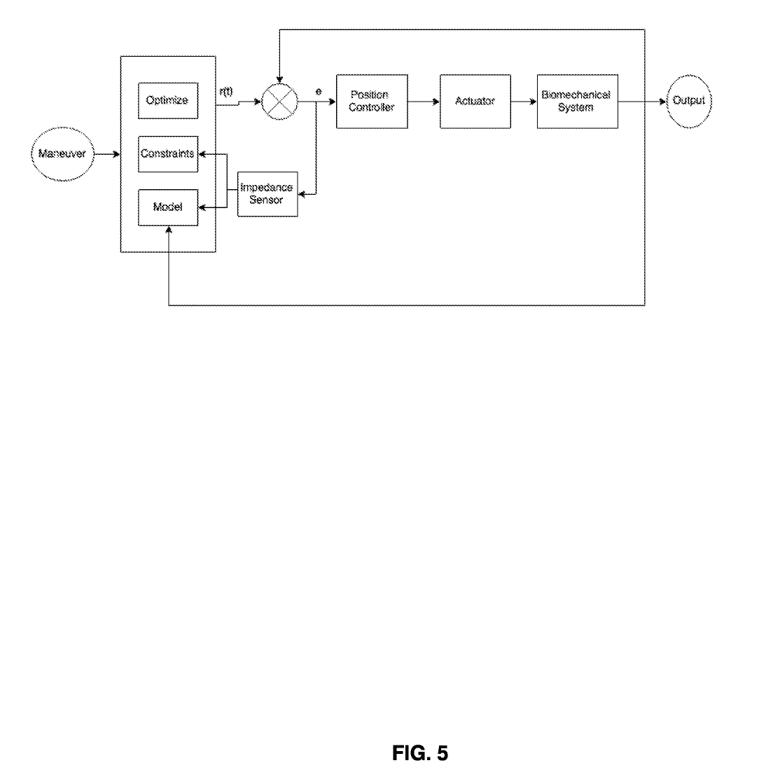

The motors and encoders (not shown, attach to the ends of the motors) all have wires that lead down to a control station. This includes a microprocessor, display, user interface, and optionally a wireless transmission device. The microprocessor collects the sensor data and outputs a command to the motors based on the control flow chart seen in FIG. 5.

This embodiment demonstrates the intraoral engagement. In an embodiment, it could be fit to engage externally or with some combination.

In multiple embodiments, the encoders can be placed in various locations with or without gearing solutions to provide other configurations.

The pivot joint, 106, can also contain a bearing to allow for rotation from the vertical linear actuator's push rod. Some actuators use a threaded rod and rotate it to produce linear motion. A similar bearing could be used in the bottom rod, 109, for a rotating push rod from the horizontal actuator.

Each base plate 102 can be coupled to a vertical linear actuator 105 and a sagittal linear actuator 105. The sagittal linear actuator 105 can be coupled to a rotating motor casing 110 that is attached to the base plate 102. The rotating motor casing 110 can allow for the sagittal linear actuator 105 to pivot along a sagittal plane. The sagittal linear actuator 105 can be coupled to a dynamic mandible support 109. The dynamic mandible support can be coupled to a bottom mouth plate 107. The bottom mouth plate 107 can be separated to allow independent operation of each side of the device. The vertical linear actuator 105 can be fixed to the base plate 102. The vertical linear actuator 105 can be coupled to a rotational joint 106 through which the dynamic mandible support 109 is capable of moving through. In an embodiment, the vertical linear actuator 105, sagittal linear actuator 105, rotational joint 106, rotating motor casing 110, and dynamic mandible supports 109 can form a dynamic actuation mechanism for the device.

To open a user's mouth, the vertical linear actuator 105 can extend and push on the rotational joint 106, moving the bottom mouth plate 107 down as the rotating motor casing 110 pivots. Similarly, to close a user's mouth, the vertical linear actuator 105 can retract, allowing the mouth to close. To protrude a user's mouth, the sagittal linear actuator 105 can extend and push out the dynamic mandible support 109; to retrude a user's jaw, the sagittal linear actuator 105 can retract the dynamic mandible support 109. Both the vertical linear actuator 105 and the sagittal linear actuator 105 can operate in tandem to produce movements in the sagittal plane.

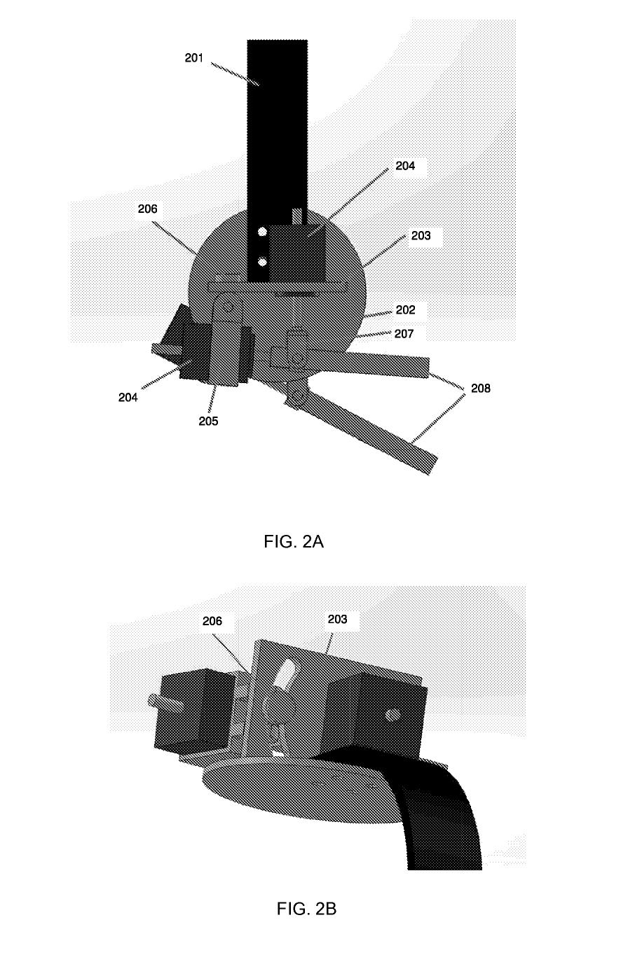

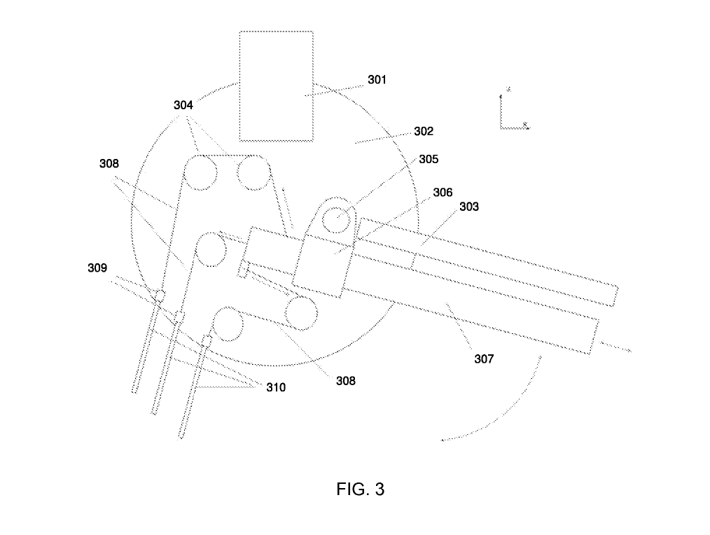

FIG. 2A and FIG. 2B are exemplary diagrams of a maxillofacial rehabilitation device having a local actuation mechanism, external mandible engagement, and three planes (sagittal, vertical, and transverse) of mandible movement, according to embodiments of the disclosure. A headset 201 can be coupled to a left and right base plate 202. A transverse plate 203 can be coupled to a base plate 202. A vertical actuator 204 can be coupled to the horizontal plate 203 and a pivot joint 207. The pivot joint can be coupled to a mandible support 208 to allow for vertical pivot of the mandible support 208. A sagittal actuator 204 can be coupled to the transverse plate with a vertical joint and transverse pivot structure, allowing the mandible support 208 to move in vertical, sagittal, and transverse planes. In an embodiment, a transverse actuator can be coupled to the sagittal actuator 204 and configured to move the mandible support in a transverse plane.

The views in FIGS. 2A and 2B show a possible 6 DOF embodiment without the outer casing or mandible engagement mechanism. The main difference from FIG. 1 is the ability of the horizontal linear actuator, 204, to pivot and slide about the groove in the Horizontal Attachment, 203. The pivot joint, 207, can contain a bearing to allow the joint to rotate relative to the push rod from the vertical linear actuator.

In an embodiment, another actuator could be added to the pin 206 to fully actuate the system. In an embodiment, the system can operate without the actuator but could use a position sensor to add accuracy to the algorithm.

The 6 DOF embodiment also demonstrates the ability to engage the mandible and maxilla externally. By adding a chin support or other engagement mechanism and coupled with the headband engaging the top of the skull, the device can actuate the mandible relative to the maxilla. This provides advantages in hygiene, lack of choking, and less contact with sensitive tissues.

FIG. 3 is an exemplary diagram of a maxillofacial rehabilitation device having a remote actuation mechanism and two planes (sagittal and vertical) of mandible movement, according to embodiments of the disclosure. A headset 301 can be coupled to a base plate 302. The base plate 302 can be coupled to a vertical pivot joint 305 and a maxilla support 303. The vertical pivot joint 305 can be coupled to a mandible support sleeve 306 that guides the mandible support 307 movement. A system of pulleys 304 can be coupled to the base plate. In an embodiment, cables 309 can be engaged with the pulleys and coupled with a remote actuator at one end and the mandible support 307 at another end. The remote actuator can control the tension of the cables 309 to effectuate movement of the mandible support 307 along sagittal and vertical planes. In an embodiment, the remote actuator can also house a controller for controlling the movement of the mandible support 307.

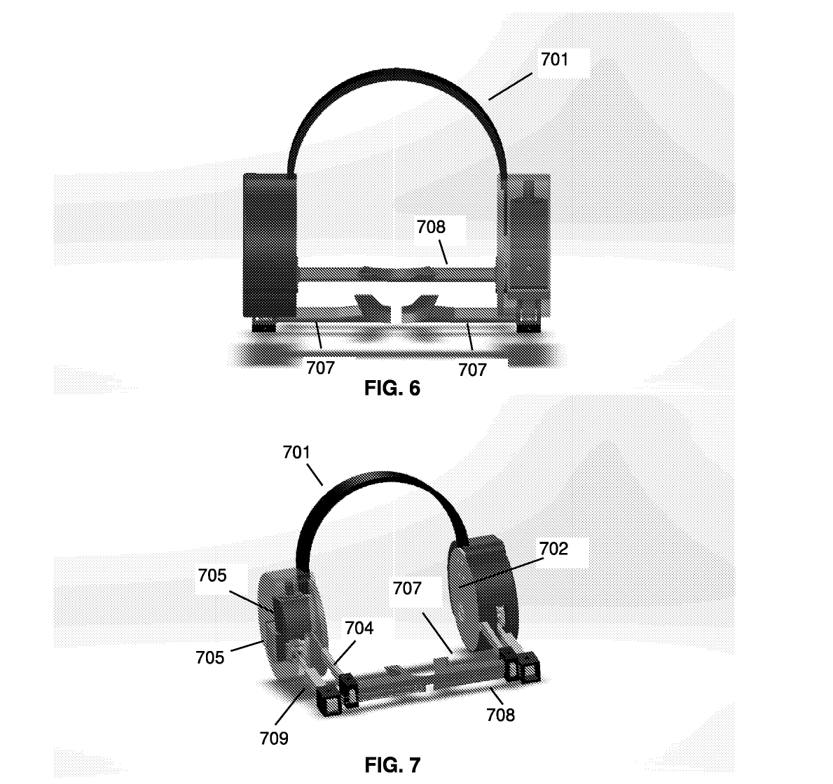

This embodiment shows the capability of placing the actuators outside of the wearable portion of the device. By retracting the cables in various patterns, the mandible support 307, can move forward and backward through the mandible support sleeve 306, while pivoting about the vertical pivot joint 305. Other views with slight variations are depicted in FIGS. 6-15.

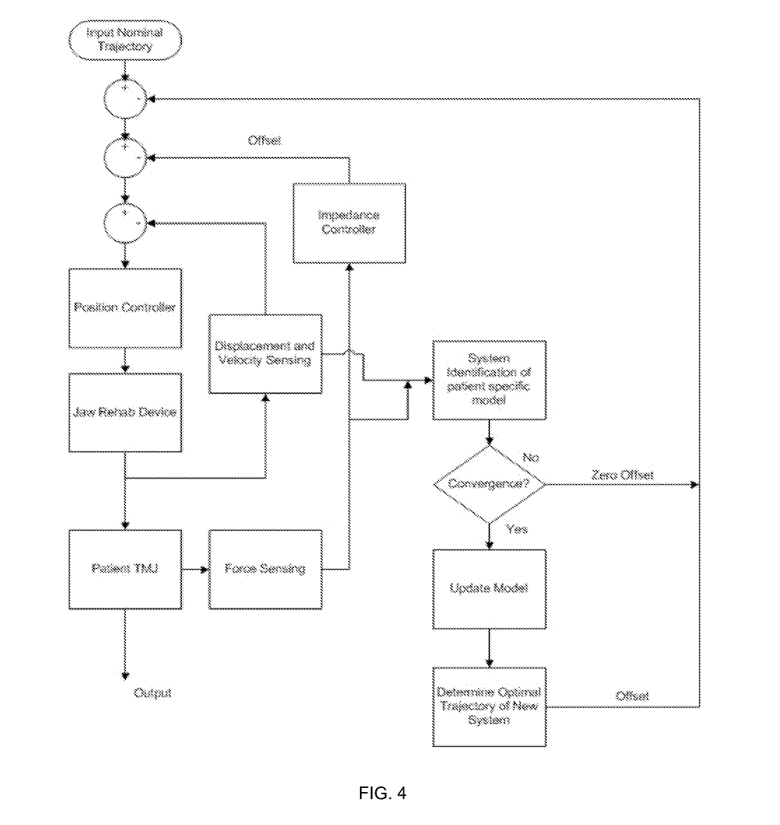

FIG. 4 is an exemplary flow diagram of a process for controlling a maxillofacial rehabilitation device, according to embodiments of the disclosure. A nominal trajectory can be input into the device. As the device is actuated by a position controller, user characteristics such as the force, displacement, and velocity of the mandible support are sensed. In an embodiment, the user characteristics can be used to identify and create a patient specific model. The controller can modify the trajectory of the device and receive feedback as to the patient specific model, updating the model as necessary. The controller can determine an improved or optimal trajectory for a patient and actuate the device according to this trajectory.

FIG. 5 is an exemplary flow diagram of a process for use of the maxillofacial rehabilitation device.

FIG. 6 is an exemplary diagram of a maxillofacial rehabilitation device. The device includes but is not limited to a headset 101, side bottom mouth plate (right and left) 707, and a top mouth plate 708.

FIG. 7 is an exemplary diagram of a maxillofacial rehabilitation device. The device includes but is not limited to a headset 701, base plate 702, maxilla bar 704, linear actuator 705, side bottom mouth plate 707, top mouth plate 708, and bottom rod 709.

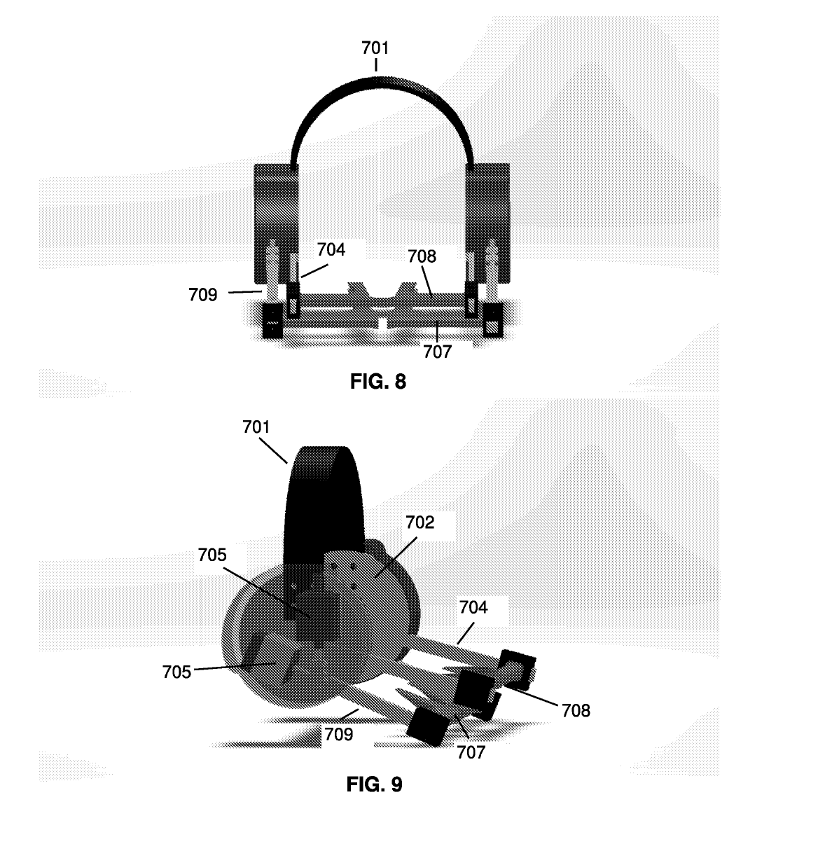

FIG. 8 is an exemplary diagram of a maxillofacial rehabilitation device. The device includes but is not limited to a headset 701, maxilla bar 704, side bottom mouth plate 707, top mouth plate 708, and bottom rod 709.

FIG. 9 is an exemplary diagram of a maxillofacial rehabilitation device. The device includes but is not limited to a headset 701, base plate 702, maxilla bar 704, linear actuator 705, side bottom mouth plate 707, top mouth plate 708, and bottom rod 709.

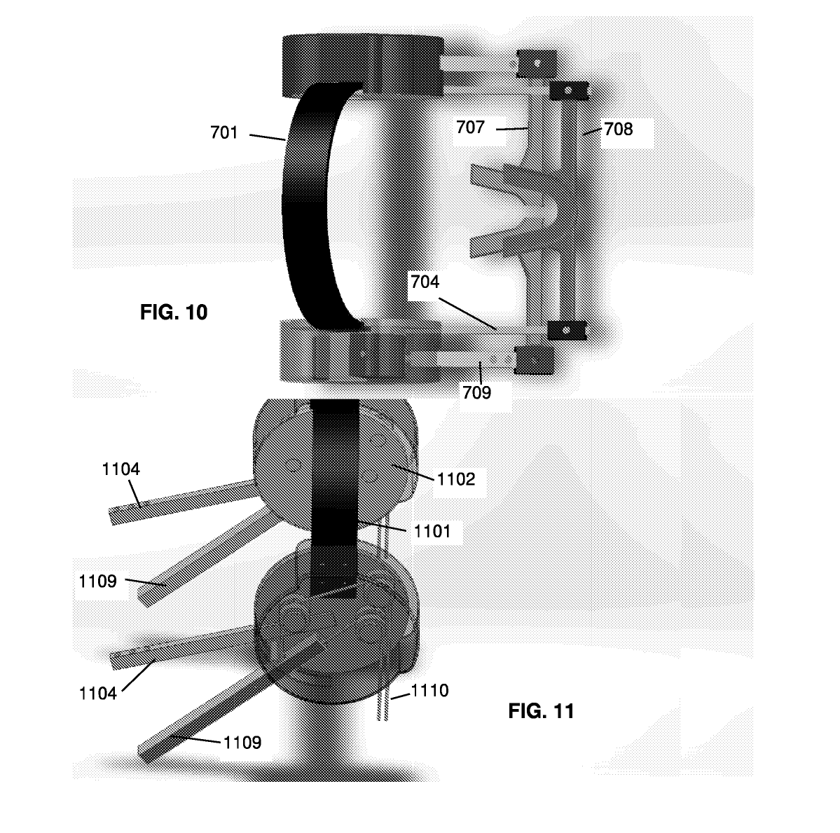

FIG. 10 is an exemplary diagram of a maxillofacial rehabilitation device. The device includes but is not limited to a headset 701, maxilla bar 704, side bottom mouth plate 707, top mouth plate 708, and bottom rod 709.

FIG. 11 is an exemplary diagram of a maxillofacial rehabilitation device. The device includes but is not limited to a headset 1101, base plate 1102, maxilla bar 1104, bottom rod 1109, and cables 1110.

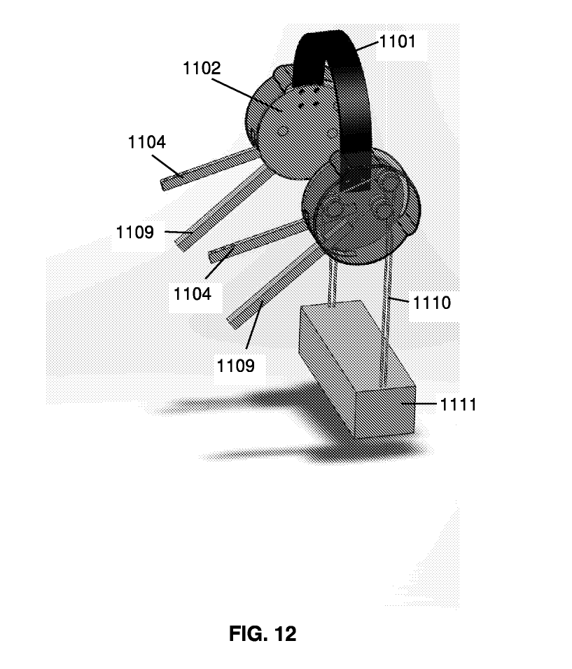

FIG. 12 is an exemplary diagram of a maxillofacial rehabilitation device having a pulley-like embodiment with motors in the external casing. The device includes but is not limited to a headset 1101, base plate 1102, maxilla bar 1104, bottom rod 1109, cables 1110, and weight 1111.

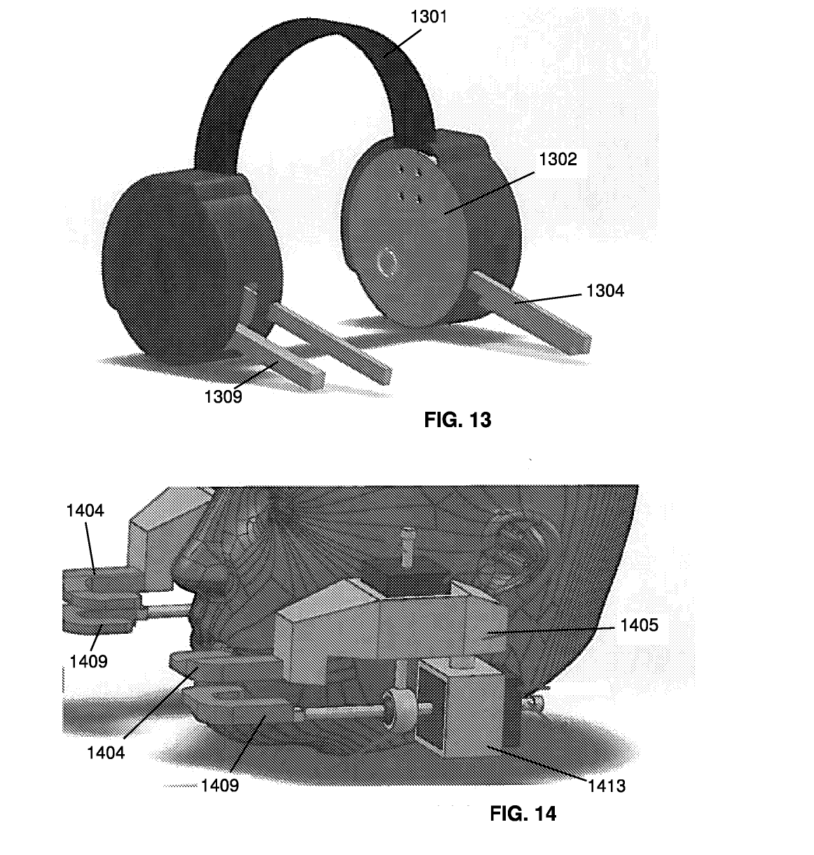

FIG. 13 is an exemplary diagram of a maxillofacial rehabilitation device having a headphone design without mouthpieces. The device includes but is not limited to a headset 1301, base plate 1302, maxilla bar 1304, and bottom rod 1309.

FIG. 14 is an exemplary diagram of a maxillofacial rehabilitation device having possible six degrees of freedom. The device includes but is not limited to a headset 1401, base plate 1402, maxilla mouthpiece 1404, linear actuator 1405, bottom mouthpiece 1409, and a linear stepper motor 1413.

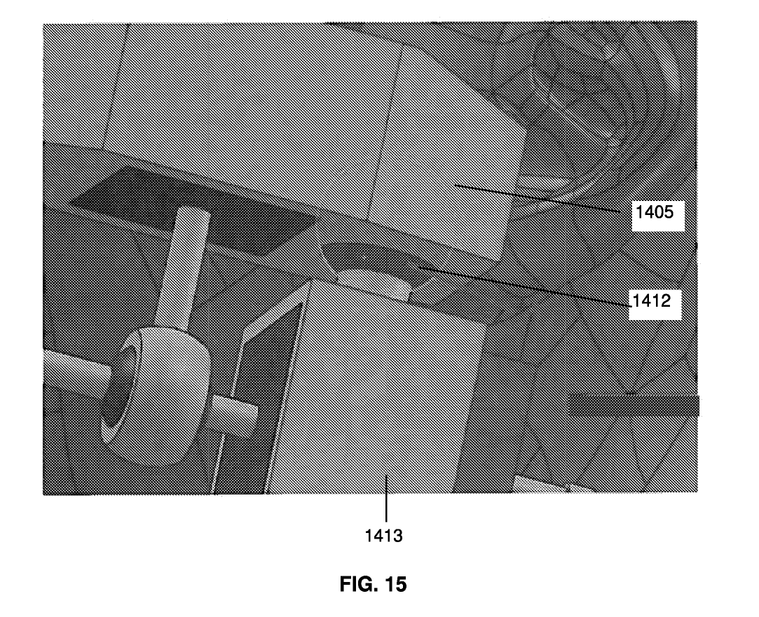

FIG. 15 is a close-up of a ball joint of FIG. 14. The device includes but is not limited to a linear actuator 1405, ball joint 1412, and a linear stepper motor 1413.

Although the present invention has been described in terms of specific embodiments, it is anticipated that alterations and modifications thereof will become apparent to those skilled in the art. Therefore, it is intended that the following claims be interpreted as covering all such alterations and modifications as fall within the true spirit and scope of the invention. All of the compositions and methods disclosed and claimed herein can be made and executed without undue experimentation in light of the present disclosure. While the compositions and methods of this disclosure have been described in terms of preferred embodiments, it will be apparent to those of skill in the art that variations can be applied to the compositions and methods and in the steps or in the sequence of steps of the methods described herein without departing from the concept, spirit and scope of the disclosure. More specifically, it will be apparent that certain components which are related can be substituted for the components described herein while the same or similar results would be achieved. All such similar substitutes and modifications apparent to those skilled in the art are deemed to be within the spirit, scope and concept of the disclosure as defined by the appended claims.

### REFERENCES

Wang, X. Dissertation, A Novel Wearable Assistive Device for Jaw Motion Disability Rehabilitation, Massey University (2014).

What is claimed is:

1. A maxillofacial rehabilitation device, comprising:
a frame, wherein the frame is used to attach the device to the head of a user;
a dynamic actuation mechanism coupled to the frame wherein the dynamic actuation mechanism
contacts the mandible of the user;
receives control signals from a controller; and
moves the mandible of the user in the sagittal plane and the vertical plane based on the control signals.

2. The device of claim 1, wherein the frame comprises a headset having a left end coupled to a left base plate and a right end coupled to a right base plate.

3. The device of claim 2, wherein the left base plate fits over a left ear of the user and the right base plate fits over a right ear of the user.

4. The device of claim 2, further comprising a maxilla plate coupled to the left base plate and the right base plate wherein the maxilla plate contacts the maxilla of the user.

5. The device of claim 4, wherein the maxilla plate contacts the inside of the mouth of the user.

6. The device of claim 2, wherein the dynamic actuation mechanism comprises
a left mandible plate coupled to a left mandible support;
a right mandible plate coupled to a right mandible support;
a left actuator mechanism coupled to the left base plate and the left mandible support and wherein the left actuator mechanism moves the left mandible plate; and
a right actuator mechanism coupled to the right base plate and the right mandible support and wherein the right actuator mechanism moves the right mandible plate.

7. The device of claim 6, wherein
the left actuator mechanism further comprises
a first left actuator, wherein the first left actuator elevates and depresses the left mandible plate;
a second left actuator, wherein the second left actuator protrudes and retrudes the left mandible plate; and
the right actuator mechanism further comprises
a first right actuator, wherein the first right actuator elevates and depresses the right mandible plate;
a second right actuator, wherein the second right actuator protrudes and retrudes the right mandible plate.

8. The device of claim 7, wherein:
the first left actuator is fixed to the left base plate;
the second left actuator is coupled to the left base plate with a left vertical pivot joint;
the first right actuator is fixed to the right base plate; and
the second right actuator is coupled to the right base plate with a right vertical pivot joint.

9. The device of claim 6, wherein the dynamic actuation mechanism moves the left mandible plate and the right mandible plate in the transverse plane.

10. The device of claim 7, wherein the frame further comprises
a left transverse plate coupled to the left base plate;
a right transverse plate coupled to the right base plate;
the left actuator mechanism further comprises a third left actuator to move the left mandible plate in the transverse plane;
the right actuator mechanism further comprises a third right actuator to move the right mandible plate in the transverse plane;
the first left actuator is coupled to the left transverse plate with a left transverse pivot joint;
the second left actuator is coupled to the left transverse plate with a left vertical pivot joint and a left transverse pivot structure;
the first right actuator is coupled to the right base plate with a right transverse pivot joint; and
the second right actuator is coupled to the right transverse plate with a right vertical pivot joint and a right transverse pivot structure.

11. The device of claim 7, wherein the first left actuator and the first right actuator move the mandible plate in the transverse plane.

12. The device of claim 6, wherein
the left actuator mechanism further comprises:
a left pulley set coupled to the left base plate and the left mandible support;
a left remote actuator coupled to the left pulley set with left cables, wherein the left cables are attached to the left mandible support; and
a left support system coupled to the left base plate and to guide the left mandible support; and
the right actuator mechanism further comprises:
a right pulley set coupled to the right base plate and the right mandible support;
a right remote actuator coupled to the right pulley set with right cables, wherein the right cables are attached to the left mandible support; and
a right support system coupled to the right base plate and to guide the right mandible support.

13. The device of claim 1, further comprising the controller sending control signals to the dynamic actuation mechanism.

14. The device of claim 13, wherein the controller senses user characteristics and determines a profile for the user based on the user characteristics; and determines control signals based on the profile.

15. The device of claim 14, wherein:
the user characteristics include mandible force and displacement information;
the control signals include left mandible plate and right mandible plate displacement, force, and velocity information.

16. The device of claim 15, wherein the controller updates the profile based on one or more changes in the user characteristics.

17. The device of claim 16, wherein:
the left actuator mechanism further comprises:
a left position sensor coupled to the controller to sense the position of the left mandible support;
a left force sensor coupled to the controller to sense the force on the left mandible support; and
a left velocity sensor coupled to the controller to sense the velocity of the left mandible support; and
the right actuator mechanism further comprises:
a right position sensor coupled to the controller to sense the position of the right mandible support;
a right force sensor coupled to the controller to sense the force on the right mandible support; and
a right velocity sensor coupled to the controller to sense the velocity of the right mandible support.

18. The device of claim 17 wherein the controller comprises a computing station connected to the device.

19. The device of claim 18 wherein the controller comprises an external user display connected to the device.

20. The device of claim 17 further comprising a wireless transmission apparatus for sending and receiving signals from the device.
